(19) United States
(12) Patent Application Publication
(10) Pub. No.: US 2005/0186187 A1
(43) Pub. Date: Aug. 25, 2005

(54) Methods for protein production

(76) Inventor: Haimanti Dorai, Exton, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/056,744

(22) Filed: Feb. 11, 2005

Related U.S. Application Data
(60) Provisional application No. 60/545,839, filed on Feb. 19, 2004.

(51) Int. Cl. A61K 48/00; A61K 39/395; C12N 5/06; C12N 15/85

(52) U.S. Cl. 424/93.21; 424/155.1; 435/326; 435/69.1; 435/455; 530/388.8

(57) ABSTRACT

Methods for altering the cellular secretion rate of a protein, such as an antibody and the altered cells produced by the method are disclosed. The methods and altered cells are useful for producing high levels of proteins for therapeutic, diagnostic or research purposes.

### CROSS REFERENCE TO RELATED APPLICATIONS

This application claims the benefit of U.S. Provisional Application No. 60/545,839, filed 19 Feb. 2004.

### FIELD OF THE INVENTION

This invention relates to methods for altering the cellular secretion rate of a protein.

### BACKGROUND OF THE INVENTION

Large-scale production of proteins, such as antibodies, typically relies on secretion of the protein from a cultured cells can be readily recovered and purified from the surrounding cell culture media.

The cellular expression rate of proteins is an important parameter affecting the production and purification of secreted proteins from a bioreactor or other system. In general, higher purified protein yields can be attained when the cellular expression rate is relatively high. Conversely, if the cellular expression rate is too low protein purification may not be feasible.

One approach to circumventing the problem of low expressing cells has been to isolate high expressing, subcloned cells from a population of low expressing cells. Typically, this requires several time-consuming and labor-intensive rounds of limiting serial dilution, screening and selection of high expressing cell lines. Alternatively, entirely new cell lines producing the protein of interest are generated in the hope that the new cell lines will be high expressing lines.

Each of the foregoing approaches to generating high expressing cell lines has limitations. For example, identifying high expressing cell lines by subcloning from a population of low expressing cells is limited by the relative rarity of high expressing cells in the population as well as the extensive amounts of time and labor required for the identification of any high expressing cells.

Further, the generation of new cell lines producing the antibody or protein of interest is limited by the possibility that the new cell lines will not be high expressing and the substantial amounts of effort that will be required to regenerate antibody producing cells and identify high expressing cells. In some instances, only low expressing cell lines can be obtained despite efforts to obtain high expressing cell lines.

Thus, a need exists for effective methods of changing the cellular secretion rate of a protein.

### BRIEF DESCRIPTION OF THE DRAWINGS

FIG. 1 shows increased SLPI gene transcript levels in high expressing cell lines relative to the Sp2/0 parent myeloma cell line.

FIG. 2 shows increased CD53 gene transcript levels in high expressing cells lines relative to the Sp2/0 parent myeloma cell line.

FIG. 3 shows increased Transferrin-1 production in high expressing cell lines relative to the Sp2/0 parent myeloma cell line.

FIG. 4 shows increased SLPI gene transcript levels in high expressing cell lines relative to the C463a parent myeloma cell line.

FIG. 5 shows the trend of increasing SLPI gene transcript levels as antibody production increases in C463a derived subclones

FIG. 6 shows increased transferrin-1 gene transcript levels in high expressing cell lines relative to the C463a parent myeloma cell line.

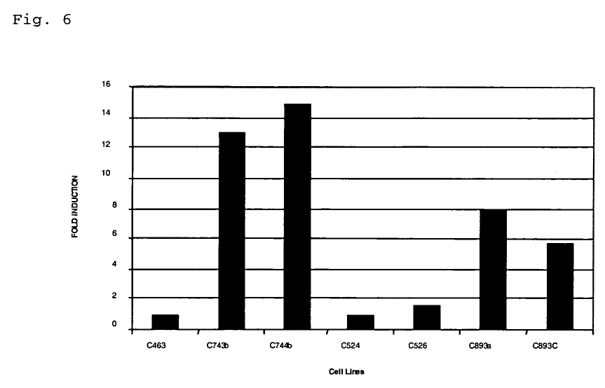

FIG. 7 shows the trend of increasing transferrin-1 gene transcript levels as antibody production increases in C463a derived subclones.

### SUMMARY OF THE INVENTION

One aspect of the invention is a method for altering the cellular secretion rate of a protein comprising the steps of modulating the activity of at least one molecule selected from the group consisting of secretory leukocyte protease inhibitor (SLPI), CD53, or Transferrin-1 in a cell and culturing the cells.

Another aspect of the invention is a myeloma cell with an altered cellular secretion rate generated by the steps of modulating the activity of at least one molecule selected from the group consisting of secretory leukocyte protease inhibitor (SLPI), CD53, or transferrin-1 in a cell; and culturing the cell.

### DETAILED DESCRIPTION OF THE INVENTION

All publications, including but not limited to patents and patent applications, cited in this specification are herein incorporated by reference as though fully set forth.

The term “antibody” as used herein is meant in a broad sense and includes immunoglobulin or antibody molecules including polyclonal antibodies, monoclonal antibodies including murine, human, humanized and chimeric monoclonal antibodies and antibody fragments or variants. Antibodies are secreted proteins constitutively expressed and secreted by plasma cells. Antibodies can also be produced using plasma cells immortalized by standard methods such as hybridoma generation or by transfection of antibody heavy and/or light chain genes into an immortalized B cell such as a myeloma cell or other cell types such as Chinese hamster ovary (CHO) cells, plant cells and insect cells.

Antibody fragments or variants include mimetibodies, Fab fragments, F(ab′)₂ fragments, Fc fragments, heavy chain fragments, light chain fragments, and molecules containing a portion of at least one antibody peptide chain. Such portions may correspond to antibody variable, hinge, or constant region peptide chains.

The term “mimetibody” as used herein means a protein having the generic formula (I):
(V1(n)-Pep(n)-Flex(n)-V2(n)-pHinge(n)-CH2(n)-CH3(n))(m) (I)
where V1 is at least one portion of an N-terminus of an immunoglobulin variable region, Pep is at least one bioactive peptide that binds to an epitope, Flex is polypeptide that provides structural flexiblity by allowing the mimetibody to have alternative orientations and binding properties, V2 is at least one portion of a C-terminus of an immunoglobulin variable region, pHinge is at least a portion of an immunoglobulin hinge region, CH2 is at least a portion of an immunoglobulin CH2 constant region and CH3 is at least a portion of an immunoglobulin CH3 constant region, where n and m can be an integer between 1 and 10. A mimetibody can mimic properties and functions of different types of immunoglobulin molecules such as IgG1, IgG2, IgG3, IgG4, IgA, IgM, IgD and IgE dependent on the heavy chain constant domain amino acid sequence present in the construct.

The term “monoclonal antibody” (mAb) as used herein means an antibody (or antibody fragment) obtained from a population of substantially homogeneous antibodies. Monoclonal antibodies are highly specific, typically being directed against a single antigenic determinant. The modifier “monoclonal” indicates the substantially homogeneous character of the antibody and does not require production of the antibody by any particular method. For example, murine mAbs can be made by the hybridoma method of Kohler et al., Nature 256: 495 (1975). Chimeric mAbs containing a light chain and heavy chain variable region derived from a donor antibody (typically murine) in association with light and heavy chain constant regions derived from an acceptor antibody (typically another mammalian species such as human) can be prepared by the method disclosed in U.S. Pat. No. 4,816,567. Humanized mAbs having CDRs derived from a non-human donor immunoglobulin (typically murine) and the remaining immunoglobulin-derived parts of the molecule being derived from one or more human immunoglobulins, optionally having altered framework support residues to preserve binding affinity, can be obtained by the techniques disclosed in Queen et al., Proc. Natl Acad Sci (USA), 86: 10029-10032, (1989) and Hodgson et al., Bio/Technology, 9: 421, (1991).

Fully human mAbs lacking any non-human sequences can be prepared from human immunoglobulin transgenic mice by techniques referenced in, e.g., Lonberg et al., Nature 368: 856-859, (1994); Fishwild et al., Nature Biotechnology 14: 845-851, (1996)′ and Mendez et al., Nature Genetics 15: 146-156, (1997). Human mAbs can also be prepared and optimized from phage display libraries by techniques referenced in, e.g., Knappik et al., J. Mol. Biol. 296: 57-86, (2000) and Krebs et al., J. Immunol. Meth. 254: 67-84, (2001).

The term “cellular expression levels” as used herein means the amount of a given protein a cell is able to express over its lifetime. Such amounts may be described as the change in the amount of protein present in the culture media per change in time (i.e. “volumetric productivity”) or can be normalized to cell number (i.e. “specific productivity”). “Volumetric productivity” can be expressed with the units “mg/ml/day” while “specific productivity” can be expressed with the units “pg/cell/day.”

The present invention provides methods useful for altering the amount of cellular expression of a protein by a cell. An exemplary use of the methods of the invention is enhancement of expression amounts for proteins that are useful for therapeutic, diagnostic or research purposes, such as antibodies.

High throughput cDNA microarray analyses provide a technique for identifying genes that are commonly modulated in different high expressing, antibody producing cell lines. Such analyses revealed that less than 0.1% of all known murine genes are commonly modulated in different SP2/0 and C463a derived murine myeloma cell lines with high secretion rates. The genes encoding the Secretory Leukocyte Protease Inhibitor (SLPI) (Genbank accession no. NM 011414), CD53 (Genbank Accession No. NM 007651 and Transferrin-1 (Genbank Accession No. J03299) proteins belong to this select set of commonly modulated genes. In cDNA microarray analyses these genes were found to be upregulated in the SP2/0 and C463a derived high expressing cell lines examined. These genes were all up-regulated in the high expressing cell lines examined by at least 1.5 fold relative to the parent murine myeloma cell lines.

The SLPI protein enhances cell proliferation by inducing cyclin D, down regulating TGF-beta, and inducing signaling through the Ras signal transduction pathway by repressing the gene encoding lysyl oxidase. The CD53 protein is a cell surface expressed member of the tetraspanin family of proteins and appears to be capable of triggering a survival response and reducing the number of cells that enter apoptosis. Other functions for CD53 such as cell activation, ion channel formation, and transport of small molecules have also been suggested. Transferrin-1 is the major iron transport protein which provides iron necessary to support cellular proliferation. While not wishing to be bound to any particular theory, the applicants believe that upregulation of one or more of the SLPI, CD53 and Transferrin-1 genes increas antibody expression by increasing viable cell numbers.

In a method of the invention, the cellular expression rate of a protein is altered by modulating the activity of at least one molecule selected from the group consisting of SLPI, CD53 or Transferrin-1 in a cell and culturing the cell. The method of the invention provides for increasing or decreasing the cellular expression rate of a protein such as an antibody.

In an embodiment of the invention, the cellular expression rate of a protein is increased by transfecting the cell with a nucleic acid encoding SLPI, CD53, or Transferrin-1. Transfection can be accomplished by standard methods such as, for example, lipofection or electroporation, or viral transformation known by those skilled in the art. Such methods can produce stably or transiently transfected cells.

Variants of the SLPI, CD53 or Transferrin-1 protein or nucleic acid sequences which produce an activity similar to the SLPI, CD53, or transferrin-1 parent molecules will also be useful in the methods of the invention. For example, variant molecules having at least 80%% identity to a parent molecule or related families of proteins would be expected to have similar activity. Percent identity between two protein sequences can be determined using the BLASTP algorithm with filtering turned off and all other default settings unchanged. Different isoforms of a polypeptide, dominant negative versions of a polypeptide, or covalently modified forms of a polypeptide are some examples of variants of a parent molecule.

In another embodiment of the invention, the cellular secretion rate of a protein can be decreased by decreasing the expression or activity of a SLPI, CD53, or transferrin-1 molecule. Expression or activity of these molecules can be decreased by administering to the cell an interfering RNA (iRNA) molecule. iRNA molecules may, for example, be short interfering RNAs (siRNAs) or antisense molecules. Methods, such as transfection techniques, for administering iRNA molecules are well known to those skilled in the art.

In the methods of the invention, exemplary cells are plasma cells, i.e., differentiated B-cells capable of expressing antibodies. Typically, the plasma cells have been immortalized by standard techniques such as viral infection, with Epstein-Barr Virus or other methods such as radiological or chemical mutagenesis. The immortalized plasma cells can also be cancerous and can be obtained by injecting mineral oil or another compound, into the peritoneal cavity of an animal.

In one embodiment of the invention, the plasma cells are what are known in the art as “myeloma cells.” In the art the term “myeloma cells” refers both to cancerous plasma cells obtained, or derived, from an organism with multiple myeloma and to hybridoma cells formed from the fusion of such a cancerous plasma cell with another cell (e.g. an antibody producing BALB/c mouse spleen cell or eukaryotic cell stably transfected with a nucleic acid encoding an antibody). Examples of myeloma cell lines include the SP2/0 (American Type Culture Collection (ATCC), Manasas, Va., CRL-1581), NSO (European Collection of Cell Cultures (ECACC), Salisbury, Wiltshire, UK, ECACC No. 85110503) and Ag653 (ATCC CRL-1580) cell lines which were obtained from mice. An example of a myeloma cell line obtained from humans is the U266 cell line (ATTC CRL-TIB-196). The C463a myeloma cell line is an example of an SP2/0 derived cell line capable of growing in chemically defined media. Those skilled in the art will recognize other myeloma cell lines.

Myeloma cells may be used to produce subclones or hybridomas capable of producing a protein such as an antibody. Subcloning may be accomplished by limiting serial dilution or other techniques well known in the art. Hybridomas may be obtained, for example, by the method of Kohler et al., Nature 256: 495 (1975) or other techniques known in the art. An antibody can be produced by subclones or hybridomas which comprise nucleic acid sequences encoding an antibody. Such nucleic acid sequences may be integrated into the chromosomal DNA, or present extrachromosomally in antibody producing subclones or hybridomas.

In one embodiment of the invention the myeloma cells are stably transfected with a nucleic acid, such as a DNA sequence. Stably transfected myeloma cells may be generated by methods of transfection, screening and selection well known to those of ordinary skill in the art. DNA sequences used to stably transfect the cells may be randomly integrated into the DNA of a myeloma cell or integrated in a site-specific manner. Such DNA sequences may encode SLPI, CD53 or Transferrin-1 molecules or iRNA capable of decreasing SLPI, CD53 or Transferrin-1 activity.

In the methods of the invention, the cells are cultured. Cells may be cultured in suspension or as adherent cultures. Cells may be cultured in a variety of vessels including, for example, bioreactors, cell bags, culture plates, flasks and other vessels well known to those of ordinary skill in the art. Cells may be cultured in IMDM (Invitrogen, Catalog number 12440-53) or any other suitable media including chemically defined media formulations. Ambient conditions suitable for cell culture, such as temperature and atmospheric composition, are also well known to those skilled in the art. Methods for the culture of cells are also well known to those skilled in the art.

The present invention also provides myeloma cells with changed cellular secretion rates generated by the methods of the invention.

The present invention will now be described with reference to the following specific, non-limiting Examples.

### EXAMPLE 1

### SLPI Gene Transcript Levels in High Expressing SP2/0 Derived Cell Lines

cDNA microarray analyses indicated that SLPI gene transcript levels are increased in SP2/0 derived high expressing cells relative to the parent SP2/0 myeloma cell line. To confirm this finding SLPI gene transcript levels in high expressing cell lines and parent SP2/0 myeloma cells were assessed via quantitative PCR (Q-PCR). High expressing cell lines examined included the antibody expressing C128D, C62, C379B, C466D and C524 cell lines which were derived from the murine SP2/0 myeloma cell line. Cells were cultured in media containing serum under standard conditions.

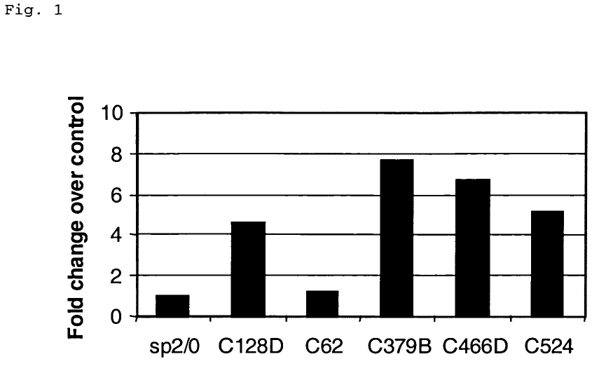

The results in FIG. 1 show that SLPI gene transcript levels are greater in SP2/0 derived high expressing cell lines relative to parent SP2/0 myeloma cells.

### EXAMPLE 2

### CD53 Gene Transcript Levels in High Expressing SP2/0 Derived Cell Lines

cDNA microarray analyses indicated that CD53 gene transcript levels are increased in SP2/0 derived high expressing cells relative to the parent SP2/0 myeloma cell line. To confirm this finding CD53 gene transcript levels in high expressing cell lines and parent SP2/0 myeloma cells were assessed via Q-PCR. High expressing cell lines examined include, in order of increasing antibody production, 175a, 175-88, and 175G, expressing 12 mg/L, 60 mg/L and 110 mg/L antibody in seven day culture, respectively. These cell lines were derived from the murine SP2/0 myeloma cell line.

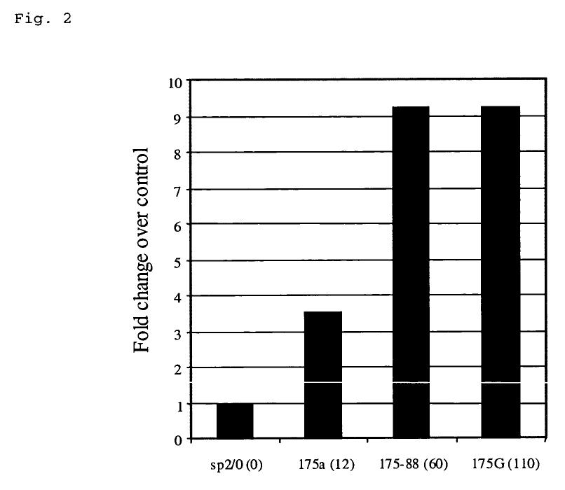

The results in FIG. 2 show that CD53 gene transcript levels are greater in SP2/0 derived high expressing cell lines relative to parent SP2/0 myeloma cells. Additionally, these results show a trend of increasing CD53 gene transcript levels as antibody production increases in the SP2/0 derived cell lines of FIG. 2.

### EXAMPLE 3

### Increased Antibody Secretion in High Expressing SP2/0 Derived Cell Lines

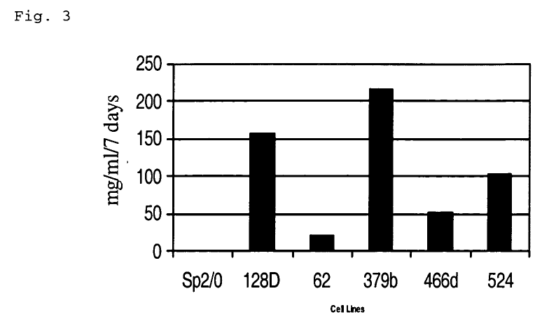

Increased antibody production occurs in high expressing SP2/0 derived cell lines relative to the SP2/0 parent myeloma cell line (FIG. 3). Comparison of FIG. 1 and 3 indicates that the fold increase in SLPI gene transcript levels appears to correlate with the rate of antibody secretion observed with the C128D, C62, C379B, C466D and C524 cell lines.

For volumetric productivity determinations, cells were seeded into fresh culture media and cultured for 7 days in a shaker flask. On day 7 the antibody concentration in the media was determined by standard assay techniques. The results in FIG. 3 represent the volumetric productivity for antibody production and are in part, a measure of the antibody secretion rate over the 7 day culture period.

### EXAMPLE 4

### SLPI Gene Transcript Levels in High Expressing C463a Derived Cell Lines

cDNA microarray analyses indicated that SLPI gene transcript levels are increased in C463a derived high expressing cells relative to the parent C463a myeloma cell line. To confirm this finding SLPI gene transcript levels in high expressing cell lines and parent C463a myeloma cells were assessed via Q-PCR. High expressing cell lines examined included the antibody expressing C743b, C744b, C524, C526, C893a, and C893c cell lines which were derived from the murine C463a myeloma cell line. The C463a myeloma cell line is an SP2/0 derived cell line capable of growing in chemically defined media. Cells were cultured in chemically defined media lacking serum under standard conditions.

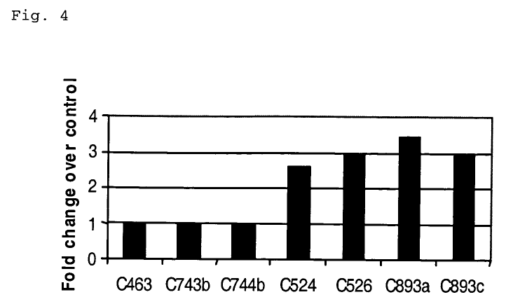

The results in FIG. 4 show that SLPI gene transcript levels are greater in the majority of C463a derived high expressing cell lines relative to parent C463a myeloma cells.

### EXAMPLE 5

### Trend of Increased SLPI Gene Transcript Levels as Subclone Antibody Production Increases in C463a Derived Cell Lines

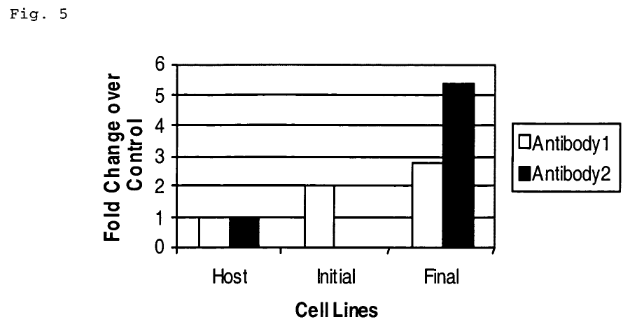

The results in FIG. 5 show a trend of increasing SLPI gene transcript levels as antibody production increases in individual C463a derived cell lines. SLPI gene transcript levels in high expressing cell lines and parent C463a myeloma cells (host) were assessed via Q-PCR. High expressing cell lines examined include, in order of increasing antibody production, a first antibody (Antibody 1) expressing “initial” cell line, the “final” first antibody producing cell line, and the “final” second antibody (Antibody 2) producing cell line. All these cell lines were derived from the murine C463a myeloma cell line. Cells were cultured in chemically defined media lacking serum under standard conditions.

### EXAMPLE 6

### Transferrin-1 Gene Transcript Levels in High Expressing C463a Derived Cell Lines

cDNA microarray analyses indicated that transferrin-1 gene transcript levels are increased in C463a derived high expressing cells relative to the parent C463a myeloma cell line. To confirm this finding transferrin-1 gene transcript levels in high expressing cell lines and parent C463a myeloma cells were assessed via Q-PCR. High expressing cell lines examined included the antibody expressing C743b, C744b, C524, C526, C893a, and C893c cell lines which were derived from the murine C463a myeloma cell line. Cells were cultured in chemically defined media lacking serum under standard conditions.

The results in FIG. 4 show that transferrin-1 gene transcript levels are greater in C463a derived high expressing cell lines relative to parent C463a myeloma cells.

### EXAMPLE 7

### Trend of Increased Transferrin-1 Gene Transcript Levels as Subclone Antibody Production Increases in C463a Derived Cell Lines

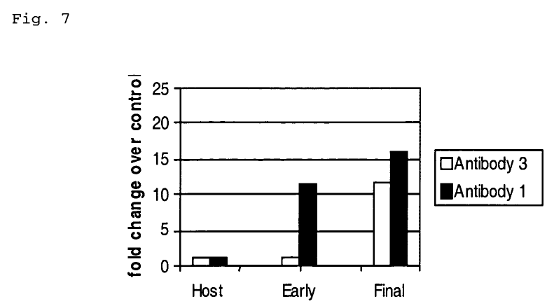

The results in FIG. 7 show a trend of increasing Transferrin-1 gene transcript levels as antibody production increases in individual C463a derived cell lines. Transferrin-1 gene transcript levels in high expressing cell lines and parent C463a myeloma cells (bars labeled “host”) were assessed via quantitative PCR (Q-PCR). High expressing cell lines examined include, in order of increasing antibody production, Antibody 1 and a third antibody (Antibody 3) expressing “early” cell lines and the “final” antibody 1 and antibody 3 producing cell lines. All these cell lines were derived from the murine C463a myeloma cell line (bars labeled “host”). Cells were cultured in chemically defined media lacking serum under standard conditions.

### EXAMPLE 8

### Effect of SLPI, CD53, and Transferrin-1 Specific Interfering RNAs on Antibody Expression Levels.

Interfering RNA molecules targeted to the SLPI, CD53, and transferrin-1 gene transcripts will alter antibody expression levels. Interfering RNA molecules can be designed using Ambion's internet based siRNA Target Finder Tool (www.ambion.com/techlib/misc/siRNA_finder.html) and can be synthesized commercially. Alternatively, permanent clones expressing siRNA transcripts can be isolated using the pSilencer™ siRNA Construction Kit (Ambion Inc., Woodward, Tex.). Interfering RNAs can be administered by the transfection of cells with nucleic acid molecules encoding interfering RNAs or the direct administration of an interfering RNA. Standard transfection techniques can be used for either approach.

### EXAMPLE 9

### Increasing Antibody Secretion Rates by Increasing SLPI, CD53, or Transferrin-1 Gene Transcript and Expression Levels

Over-expression or other techniques to increase the activity of SLPI, CD53 or transferrin-1 in cells will increase the secretion rates of proteins such as mAbs by cells. Protein expressing cell lines, such as mAb expressing cell lines, may be transfected with expression vector constructs encoding SLPI, CD53, or transferrin-1 to effect the over-expression of these proteins. Cells can be transfected with these expression vector constructs either individually or in combination. Appropriate protein and antibody expression levels may be determined after transfection using standard techniques. Protein secretion rates in transfected cells may then be compared to the secretion rates of non-transfected control cells. Protein secretion rates are expected to be higher in cells over-expressing one or more molecule from the group consisting of SLPI, CD53 or Transferrin-1.

The present invention now being fully described, it will be apparent to one of ordinary skill in the art that many changes and modifications can be made thereto without departing from the spirit or scope of the appended claims.

1. A method for altering the expression levels of a protein comprising the steps of:
a) modulating the activity of at least one molecule selected from the group consisting of secretory leukocyte protease inhibitor (SLPI), CD53, or Transferrin-1 in a cell; and
b) culturing the cells.

2. The method of claim 1 wherein the protein expression levels are increased.

3. The method of claim 2 wherein the molecule is modulated by transfecting the cell with a nucleic acid encoding SLPI, CD53, or transferrin-1.

4. The method of claim 3 wherein the nucleic acid encodes a molecule having the amino acid sequence of mouse SLP1, mouse CD53 or mouse Transferrin-1.

5. The method of claim 4 wherein the nucleic acid has the nucleotide sequence of mouse SLP1, mouse CD53 or mouse Transferrin-1.

6. The method of claim 1 wherein the cell is a myeloma cell.

7. The method of claim 6 wherein the myeloma cell is Sp2/0, NS0, Ag653, or C463a.

8. The method of claim 6 wherein the myeloma cell is a subclone or hybridoma derived from Sp2/0, NS0, Ag653, or C463a.

9. The method of claim 1 wherein the protein is an antibody.

10. The method of claim 1 wherein the cellular secretion rate is decreased.

11. The method of claim 10 wherein the molecule is modulated by administering to the cell an interfering RNA or a nucleic acid encoding interfering RNA.

12. A myeloma cell with an altered protein expression level generated by the steps of:
a) modulating the activity of at least one molecule selected from the group consisting of secretory leukocyte protease inhibitor (SLPI), CD53, or transferrin-1 in a cell; and
b) culturing the cell.
